(19) United States
(12) Patent Application Publication
(10) Pub. No.: US 2008/0039425 A1
(43) Pub. Date: Feb. 14, 2008

(54) Methods and Compositions to Reduce Tissue Irritation in Parenteral Formulations

(75) Inventor: Douglas Giles Johnson, Arvada, CO (US)

Correspondence Address: SWANSON & BRATSCHUN, L.L.C., 8210 SOUTHPARK TERRACE, LITTLETON, CO, 80120, US

(73) Assignee: ALLOS THERAPEUTICS, INC., Westminster, CO (US)

(21) Appl. No.: 11/573,063

(22) PCT Filed: Aug. 2, 2005
(86) PCT No.: PCT/US05/27397

§ 371 Date: Jun. 13, 2007

Related U.S. Application Data
(60) Provisional application No. 60/598,933, filed on Aug. 4, 2004.

(51) Int. Cl. A61K 31/715

(52) U.S. Cl. 514/58

(57) ABSTRACT

Compositions comprising a therapeutic compound and a dextin are disclosed, as well as processes for the preparation of the compositions. Also disclosed are methods using the compositions, including a method for reducing local irritation and resulting pain from an injection of a therapeutic compound by administering the composition.

### BACKGROUND

Certain drugs can cause tissue damage and or pain when administered into a vein where blood flow does not dilute the drug quickly enough. In an early study in humans receiving Efaproxiral Injection (Efaproxiral is a substituted alpha-phenoxy carboxylic acid) in a peripheral vein, some recipients complained of pain near the site of injection. In order to avoid this pain in future studies the drug was administered via a central line. The blood flow is greater in the central line and no reports of injection related events occurred. However, central lines are expensive and placement can require a surgical procedure. A way to decrease the negative interaction of a drug with tissue so that it could be administered peripherally would be desirable.

### SUMMARY OF THE INVENTION

The present invention provides a composition comprising a therapeutic compound and a dextrin. The present invention also provides a process for the preparation of a composition comprising a therapeutic compound and a dextrin comprising mixing the therapeutic compound with the dextrin.

The present invention also provides a method for the treatment of a patient in need of a therapeutic compound by administering such patient a composition comprising the therapeutic compound and a dextrin.

The present invention further provides a method for reducing local irritation and resulting pain from an injection of a therapeutic compound by administering a composition comprising the therapeutic compound and a dextrin.

### DETAILED DESCRIPTION OF THE INVENTION

The present invention provides novel compositions of matter comprising an irritation-reducing amount of a dextrin compound, including a dextrin, and a therapeutic compound. In some embodiments the dextrin is a substituted dextrin. As used herein, anti-irritation-effective amount means an amount of a substance which when combined with a compound, cytotoxic drug, antibiotic or alkaloid, with or without an excipient and administered to a subject, significantly reduces the extent of irritation that occurs, if any, compared to the extent of irritation caused by the same amount of compound, cytotoxic drug, antibiotic or alkaloid, with or without an excipient when administered alone to a subject.

As used herein, irritant is meant to include a therapeutic agent that may produce pain and or inflammation at or near the administration site or along the path of the vein (phlebitis) by which it is administered. Examples of anti-cancer chemotherapeutic agents which are irritants include but are not limited to Carmustine, Dacarbazine, Etoposide, Plicamycin, Etoposice, Streptozocin and Tenoposide.

A dextrin, as used herein, is a carbohydrate generally produced by the action on starch of acids, heat, or enzymes. Cyclical dextrins are known as cyclodextrins. As used herein, linear dextrins are dextrins that are not cyclodextrins. In general the dextrin (C₆H₁₀O₅)ₙ, is made up primarily of polymers of d-glucose linked primarily by α-(1→4) bonds but optionally having some branched segments linked by α-(1→6) bonds. The molecular weight of dextrins can be as low as several hundred or as high as 100,000. The invention includes the result that linear dextrins are as effective as cyclodextrins. The solution proposed in this invention is the inclusion of dextrins, or modified dextrins, in the formulation of drugs that cause pain or tissue damage. It is interesting to note that dextrans, which are polymers of d-glucose characterized by predominately α-(1-6) linkages do not have the desired effect.

Some compounds that are allosteric hemoglobin modifiers are irritants, and may produce pain and/or inflammation upon administration. Accordingly, the present invention is includes compositions comprising dextrin compounds and allosteric hemoglobin modifiers.

An example is efaproxiral (2-[4-[2-[(3,5-dimethylphenyl)amino]-2-oxoethyl]phenoxy]-2-methyl-propionic acid and/or its physiologically acceptably salts). The preparation and uses for 2-[4-[2-[(3,5-dimethylphenyl)amino]-2-oxoethyl]phenoxy]-2-methyl-propionic acid and its physiologically acceptable salts has been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and pending U.S. patent application Ser. No. 10/082,130, filed Feb. 25, 2002.

Efaproxiral, in the presence of dextrins, forms an efaproxiral-dextrin complex. The dextrin, by complexing with the efaproxiral, acts to shield the vein from the efaproxiral long enough for the concentration of efaproxiral in the blood to drop by several possible mechanisms: (1) dilution by the blood volume, (2) loss of efaproxiral to protein binding (such as to albumin), and (3) efaproxiral entering the red blood cells where it binds to hemoglobin.

In general, the invention provides a composition of matter comprising a complex of dextrin and any compound which can cause irritation when injected. While many such compounds are cytotoxic compounds, the compositions of matter according to the invention are not limited to cytotoxic compounds.

Compositions of matter comprising a complex of dextrin and a compound according to the invention may comprise a variety of different compounds used for a variety of therapeutic purposes. Such compositions according to the invention include a complex of dextrin and an anti-cancer, anti-neoplastic, anti-fungal antibiotic, anti-bacterial antibiotic or chemical compound.

With respect to the compositions of matter comprising a complex of dextrin and a compound which is a chemotherapeutic anticancer agent, the anticancer agent may be classified as a vesicant or an irritant. By vesicant is meant a chemotherapeutic agent which is topically toxic. If inadvertantly delivered outside of a vein, a vesicant has the potential to cause pain, cellular damage including cellulitis, tissue destruction (necrosis) with formation of a sore or ulcer and sloughing of tissues that may be extensive and require skin grafting. Examples of anti-cancer chemotherapeutic agents that are vesicants include but are not limited to, Amsacrine, Dactinomycin, Daunorubicin, Doxorubicin, Idarubicin, Mechlorethamine, Mitomycin C, RH-1 (2,5-diaziridinyl-3-hydroxymethyl-6-methyl-1,4-benzoquinone), Vinblastine, Vincristine and Vindesine. Examples of anti-cancer chemotherapeutic agents which are irritants include but are not limited to Carmustine, Dacarbazine, Etoposide, Plicamycin, Etoposide, Streptozocin and Tenoposide. Efaproxiral is an example of a compound that is not a chemotherapeutic agent but which can be irritating when delivered at high concentrations.

In general, the composition of matter according to the invention will comprise a sufficient amount of the compound to exert its desired pharmacological effect when administered IV, whether it is for example sedation, anti-fungal activity, anti-neoplastic activity, and an amount of dextrin compound sufficient to significantly reduce the extent of irritation that would occur if a like amount of the compound were administered IV in the absence of the dextrin compound. For example, the composition of matter according to the invention will comprise a sufficient amount of the anticancer compound to exert its desired cytotoxic effect against target cancer cells and anti-irritation-effective amount of dextrin with or without an excipient.

The compositions of matter according to the invention may also include, in addition to the complex of dextrin and a therapeutic compound, carriers, buffers, diluents, and other pharmaceutically acceptable excipients such as mannitol, sorbitol, lactose, sucrose and the like. With reference to efaproxiral, suitable formulations are described in copending U.S. patent application Ser. No. 10/120,848, incorporated by reference herein in its entirety.

In some embodiments of the invention, the dextrins are chemically modified or substituted. Chemical substitution at the 2,3 and 6 hydroxyl groups of the glucopyranose units of the dextrin polymer (linked 1→4) can yield increases in solubility of the dextrin compound.

In some embodiments, dextrins in the compositions according to the invention are maltodextrin compounds. By maltodextrin is meant mixtures of linear dextrins with average molecular weights from about 900-9000. Maltodextrin has the benefit of being safe, readily metabolized, and available in pharmaceutical grade (USP or EP). In some embodiments, a modified dextrin is prepared by non-selective alkylation of the desired dextrin species. Suitable alkylation agents for this purpose include but are not limited to propylene oxide, ethylene oxide, glycidol, iodoactamide, chloroacetate, and 2-diethylaminoethlychloride. Reactions are carried out to yield mixtures containing a plurality of components thereby preventing crystallization of the dextrin, various alkylated dextrins can be made and of course will vary, depending upon the starting species of dextrin and the alkylating agent used. The particular dextrin or alkylated dextrin to be used with the particular therapeutic compound to form the compositions according to the invention will be selected based on the size of the molecule of the therapeutic compound and the complex-forming abilities of the dextrin compound with the therapeutic compound. The use of a particular dextrin with a particular therapeutic compound or therapeutic compound and excipient in the compositions according to the invention may of course be optimized based on the effectiveness in reducing irritation. In some embodiments, dextrins include hydroxypropyl-maltodextrin, ethoxypropyl-maltodextrin, heptamaltose, and maltodextrin.

Another significant factor in determining the anti irritation effects of complexes of dextrins and therapeutic compounds is the degree of substitution of substituent groups in the dextrin molecule. By degree of substitution is meant the number of substituent molecules per molecule of dextrin.

As mentioned above, the compositions of matter of the invention comprise a therapeutic compound and dextrin. The relative amounts of therapeutic compound and dextrin will vary depending upon the relative toxicity of the compound and the effect of the dextrin on the compound. In general, the ratio of the therapeutic compound to the dextrin compound will be in a range between 1:0.1 to 1:20. In other embodiments, a range of 1:0.25 to 1:5 of therapeutic compound to dextrin is believed to be effective for a number of therapeutic compounds.

The compositions of matter according to the invention may be supplied as a powder or solution comprising the active pharmaceutical compound and dextrin compound. If the composition is to be administered parenterally, for example intravenously, the composition of matter will be rendered sterile prior to such administration. Any of the several known means for rendering such pharmaceutical preparations sterile may be used so long as the active pharmaceutical compound is not inactivated. If the active pharmaceutical compound is heat stable, the composition of matter according to the invention may be heat sterilized. In another alternative, the composition of matter according to the invention may be filter sterilized using, for example, a 0.2 micron filter. If the composition of matter is an aqueous liquid, it may be filled in a sterile container and supplied as a sterile liquid ready for farther dilution or injection neat. Alternatively such sterile liquids may be freeze dried or lyophilized in a sterile container and capped.

In general the compositions of matter according to the invention will be made by dissolving the dextrin in water and adding the active compound to the aqueous dextrin solution. Excipients, if any are desired, may be added with the active compound. The resulting solution may be sterilized using any of the known methods appropriate to preserving the active compound. Alternatively, the components may be sterilized by any of the known methods appropriate to preserving the active compound prior to mixing in water and may be mixed using sterile equipment and technique. Water can be removed from the reaction mixture by known methods, i.e. by freeze-drying or spray drying. For example, the solution may be lyophilized in sterile containers and capped. Prior to use the lyophilized composition of matter may be reconstituted using a sterile diluent such as water for injection, 0.9% saline or 5% dextrose.

It will be understood that the compositions of matter according to the invention provide novel methods of controlling and reducing the irritation associated with intravenous administration of many pharmaceutical compounds. The compositions according to the invention provide a method for reducing the likelihood of irritation in subjects in need of parenteral treatment with compounds that when administered parenterally, particularly intravenously, have the potential for causing irritation, by administering to such subject a preparation comprising at least one compound that has the potential for causing irritation and an anti-irritation-effective amount of dextrin.

It will be understood that the present invention provides both compositions of matter and methods for the substantial reduction in irritation and pain caused by administration of certain therapeutic compounds.

Accordingly, the present invention is directed to compositions comprising anti irritation-effective amounts of dextrin and compounds that otherwise cause pain and/or irritation when administered. Such compounds may be soluble in aqueous solution or alternatively may be lipophilic and as a result tend to precipitate in aqueous solutions. Accordingly, the invention provides compositions of matter comprising dextrin and such insoluble compounds, which have been rendered soluble by complexation with dextrin and do not promote irritation upon administration.

The invention will be better understood from the following example which is intended to be merely illustrative of the invention and are not intended to be limiting.
Specific Example of the Invention—Efaproxiral:

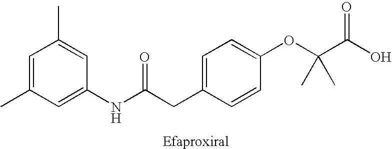

The NMR of efaproxiral is useful for detecting complexation phenomena. While there are several changes in the NMR spectrum of efaproxiral when a carbohydrate complex is formed, the most diagnostic signal in the ¹³C spectrum of efaproxiral with several compounds was that due to the two methyl groups alpha to the carboxylic acid. These two methyls are equivalent in efaproxiral and give a single signal. When complexed with, for example, cyclodextrin, the equivalence is broken and two equal signals result. Table 1 shows the splitting of this signal with several different carbohydrates in solution.

TABLE 1
NMR Data

| | Molar | Splitting in ¹³C |
|---|---|---|
| Sample | Ratio | NMR |
| Efaproxiral | — | 0 |
| Efaproxiral + trehalose | 1:1 | 0 |
| Efaproxiral + Hydroxypropyl- | 1:1 | 0.05 |
| β- | | |
| cyclodextrin | | |
| (β-HPCD) | | |
| Efaproxiral + Heptamaltose | 1:1 | 0.09 |
| (7.2%) | | |
| Efaproxiral + Maltodextrin | 1:1.4* | 0.15 |
| (10%) | | |

Calculating moles of maltodextrin using Mw as molecular weight. (All samples contained efaproxiral at 20 mg/mL, 0.225% NaCl and 1 millimolar phosphate buffer with the pH adjusted to about 7.5.)

These results show that the linear dextrins, like the cyclodextrins, form complexes with efaproxiral breaking the equivalence of the two methyl groups alpha to the acid in efaproxiral. The result for trehalose, a simple small carbohydrate dimer, shows no effect on the signal for the two methyl groups suggesting that larger dextrins are required.

In order to determine if the effects seen in the NMR correlated to an ability of the dextrins to protect tissue, an animal model was designed. Efaproxiral was tested at a series of concentrations in a rat tail vein until tissue damage was observed in the histopathological evaluation of the animal. A 38 mg/mL efaproxiral solution was administered as the positive control. A saline solution was used as the negative control. The results for several test compositions are shown in Table 2.

TABLE 2
Rat-Tail Model Data

| | Histopathology |
|---|---|
| Test Article | Score* |
| Efaproxiral 38 mg/mL | 100% |
| Efaproxiral 20 mg/mL | 24% |
| Saline | 11% |
| 38 gm/mL efaproxiral + 25% | 9% |
| Maltodextrin | |
| (1:1.8 molar ratio) | |
| 38 gm/mL efaproxiral + Dextran40 | 100% |
| 38 gm/mL efaproxiral + β-HPCD | 12% |
| (1:1 molar ratio) | |

*Relative to efaproxiral at 38 gm/mL

When efaproxiral-Na is administered at 38 mg/mL in a rat tail significant damage was observed beyond that associated with the administration (damage observed in saline control). When 250 mg/mL of maltodextrin was added to the 38 mg/mL efaproxiral solution the damage was indistinguishable from that of the negative control. The same was also true for a solution in which modified β-cyclodextrin was added at a 1:1 molar ratio. Maltodextrin has the property of being a safer molecule than its cyclic relative.

The addition of simple linear carbohydrates is able to decrease the irritation. The animal model shows that the presence of the maltodextrin, a linear carbohydrate, decreases the irritation of the efaproxiral. In addition, the NMR data support the possibility of the formation of a maltodextrin-efaproxiral complex as the mechanism of action.

What is claimed is:

1. A composition comprising a dextrin and a therapeutic compound, wherein the composition is non-irritating.

2. A composition according to claim 1, wherein the therapeutic compound is known to have an irritating property.

3. A composition according to claim 1, wherein the therapeutic compound is an allosteric hemoglobin modifier.

4. A composition according to claim 3, wherein the therapeutic compound is efaproxiral.

5. A composition according to claim 4, wherein the dextrin is maltodextrin.

6. The composition of claim 4, wherein the composition confers reduced pain upon injection in comparison to a composition of efaproxiral not comprising a dextrin.

7. A composition according to claim 1, wherein the therapeutic compound is 2,5-diaziridinyl-3-hydroxymethyl-6-methyl- 1,4-benzoquinone.

8. A composition according to claim 7, wherein the dextrin is maltodextrin.

9. The composition of claim 7, wherein the composition confers reduced pain upon injection in comparison to a composition of 2,5-diaziridinyl-3-hydroxymethyl-6-methyl-1,4-benzoquinone not comprising a dextrin.

10. The composition of claim 9 where the drug product is lyophilized during manufacture.

11. The composition according to claim 1, wherein the dextrin is present in an anti-irritation-effective amount.

12. The composition of claim 1, wherein the dextrin is a linear dextrin.

13. The composition of claim 12, wherein the dextrin is selected from the group consisting of hydroxypropyl-maltodextrin, ethoxypropyl-maltodextrin, heptamaltose, and maltodextrin.

14. A process for preparation of the composition according to claim 4 comprising mixing efaproxiral with the dextrin.

15. A method for treatment of a patient in need of efaproxiral by administering the composition according to claim 4.

16. A method for reducing local irritation and resulting pain from an injection of efaproxiral by administering the composition according to claim 4.
